(19) United States
(12) Patent Application Publication
(10) Pub. No.: US 2007/0005106 A1
(43) Pub. Date: Jan. 4, 2007

(54) Systems and methods to facilitate muscular benefit using vascular occlusion

(76) Inventor: James Paul Adducci, La Crosse, WI (US)

Correspondence Address: SCHWEGMAN, LUNDBERG, WOESSNER & KLUTH, P.A., P.O. BOX 2938, MINNEAPOLIS, MN, 55402, US

(21) Appl. No.: 11/222,460

(22) Filed: Sep. 8, 2005

Related U.S. Application Data
(60) Provisional application No. 60/695,644, filed on Jun. 30, 2005.

(51) Int. Cl. A61B 17/00

(52) U.S. Cl. 606/202

(57) ABSTRACT

Systems, devices, and methods for facilitating muscular benefit using vascular occlusion are described. A device for providing vascular occlusion to a limb of an individual is described herein. The device is comprised of an inflatable band member, a pressure-inducing device, and a pressure control device. The device is adapted to be worn over an extended period of time either concurrent with exercise or not concurrent with exercise. Additionally, methods using the device are described wherein a limited pressure is applied to the limb and then maintained over an extended period of time. Methods are also described using a device wherein the individual applies the device to their limb, applies pressure to the limb by tightening and securing the device, and leaves the device in place over an extended period of time.

This application claims the benefit of priority, under U.S.C. Section 119(e), to U.S. Provisional Patent Application Ser. No. 60/695,644, filed Jun. 30, 2005, which is incorporated herein by reference.

### FIELD OF THE INVENTION

The present invention relates generally to systems and devices that provide muscular benefit and more particularly to vascular occlusion systems and devices that provide muscular benefit.

### BACKGROUND

It is well known that aerobic exercise such as running can lead to greater endurance by improving the oxidative capacity and efficiency of Type I muscle fiber. It is also well known that anaerobic exercise such as resistance training can lead to greater size and strength through the repair of Type II muscle fiber broken down by lactic acid. Low-intensity resistance training has been considered much less effective at providing benefits to Type II muscle fiber than a higher-intensity level of resistance relative to someone's one repetition maximum.

Additionally, persons who live, exercise, and/or have ancestry in areas of higher elevation are predisposed to having greater size, strength, and endurance than individuals who live, train, and/or have ancestry in areas of lower elevation. The advantage is clear in the sports arena when individuals predisposed to higher elevations compete at lower altitudes. This disadvantage is also clear when individuals predisposed to lower elevations compete at higher altitudes. From African runners training at elevation near Mount Kilimanjaro to European weight lifters in the altitudes of Scandinavia, these athletes achieve a higher level of performance because of a reduced level of oxygen in the environments in which they live, train, and/or have been genetically predisposed to.

While everyone stands to benefit from a higher level of muscular strength and endurance, many people stand to benefit by simply maintaining the levels they currently have. Patients recovering from surgery that do not yet have the ability to use a limb at the same level of performance they could before the surgery experience muscular disuse atrophy. Astronauts in low-gravity must exercise on a regular basis and maintain use of their limbs because lack of exercise and general use has been shown to lead to increased muscular atrophy. Persons with peripheral arterial disease experience greater levels of fatigue through normal activities than persons without the disease. In order to have an acceptable quality of life, these persons need to increase their endurance and resistance to fatigue, but high-intensity exercise used to accomplish this carries with it an increased risk of negative side effects such as stroke and heart attack.

There are many individuals that do not have the ability to perform intense resistance exercise but are capable of lifting smaller loads relative to their one repetition maximum. What is needed is a way for an individual to achieve the benefits of high-intensity resistance training with lower-intensity levels of resistance.

### SUMMARY OF THE INVENTION

Systems and methods for the facilitation of muscular benefit using vascular occlusion are described. In one embodiment, a vascular occlusion device includes an inflatable band member, a pressure-inducing device, and a pressure control device. In a further embodiment, the vascular occlusion device further includes a controller coupled to the pressure-inducing device and the pressure control device. In another embodiment, a vascular occlusion system includes one or more inflatable band members, one or more pressure-inducing devices, and one or more pressure control devices. In a further embodiment, the vascular occlusion system includes a controller coupled to the one or more pressure-inducing devices and the one or more pressure control devices. In an embodiment, a method of providing muscular benefit using vascular occlusion is described, the method comprising inflating a wearable band, determining if the pressure in the inflated wearable band has reached some pre-set value, and maintaining that pressure once it has reached the pre-set value. In yet another embodiment, a method of providing muscular benefit using vascular occlusion is described, wherein the method comprises applying a non-inflatable cord to the limb of an individual, tightening the non-inflatable cord until a pressure is applied to the limb, and securing the non-inflatable cord to maintain pressure on the limb.

### BRIEF DESCRIPTION OF THE DRAWINGS

In the drawings, which are not necessarily drawn to scale, like numerals describe substantially similar components throughout the several views. Like numerals having different letter suffixes represent different instances of substantially similar components. The drawings illustrate generally, by way of example, but not by way of limitation, various embodiments discussed in the present document.

FIG. 1 is a perspective view of a device for muscular benefit using vascular occlusion, according to embodiments of the present invention;

FIG. 2A is a perspective view of a system for muscular benefit using vascular occlusion, according to embodiments of the present invention;

FIG. 2B is a perspective view of a system for muscular benefit using vascular occlusion, according to embodiments of the present invention;

FIG. 3A is a high-level block diagram of a device, such as the device depicted in FIG. 1, for muscular benefit using vascular occlusion;

FIG. 3B is a high-level block diagram of a system, such as the system depicted in FIG. 2A and FIG. 2B, for muscular benefit using vascular occlusion;

FIG. 3C is a high-level block diagram of a system, such as the system depicted in FIG. 2A and FIG. 2B, for muscular benefit using vascular occlusion;

FIG. 3D is a high-level block diagram of a system, such as the system depicted in FIG. 2A and FIG. 2B, for muscular benefit using vascular occlusion; and

FIG. 4 is a flowchart of a method to be carried out in accordance with embodiments of the present invention.

### DETAILED DESCRIPTION

In the following detailed description, reference is made to the accompanying drawings that form a part hereof and, in which is shown by way of illustration, specific embodiments in which the invention may be practiced. These embodiments are described in sufficient detail to enable those skilled in the art to practice the invention. It is to be understood that other embodiments may be utilized and that structural changes may be made without departing from the scope of the inventive subject matter disclosed herein. Therefore, the following detailed description is not to be taken in a limiting sense. The scope of the inventive subject matter disclosed herein is defined by the appended claims and their equivalents.

Overview

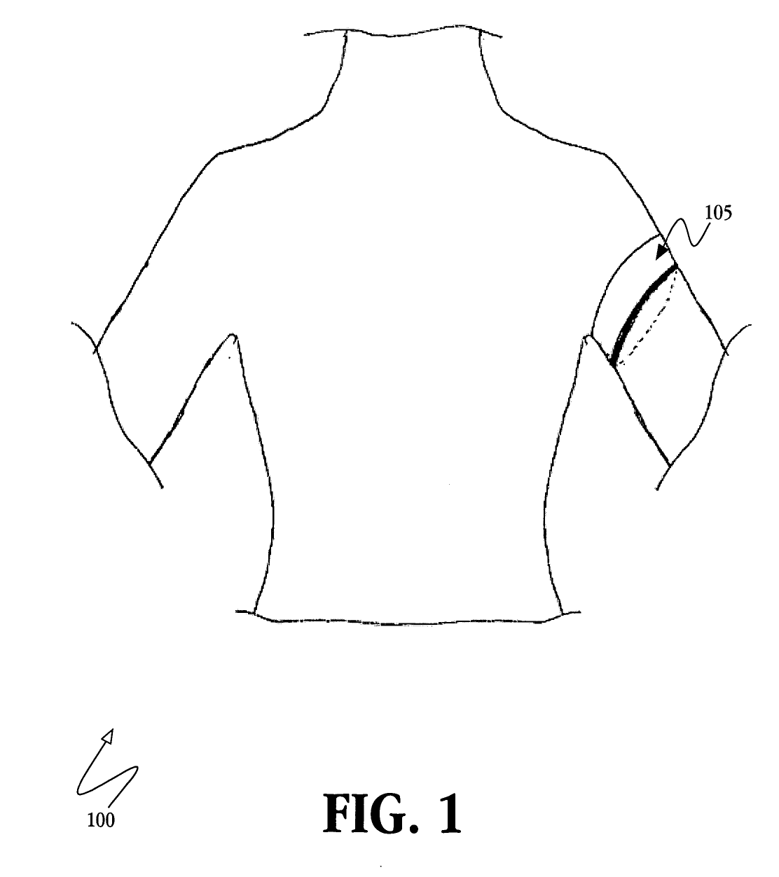

FIG. 1 is a perspective view of a system for muscular benefit using vascular occlusion, according to embodiments of the present invention. In an embodiment, the muscular benefit system 100 comprises a muscular benefit device 105 applied to the limb of an individual.

In an embodiment, the muscular benefit device 105 is configured to apply a pressure to a blood vessel on the limb of the individual. Blood vessel includes, without limitation, veins and arteries. Arteries include, without limitation, the brachial artery of the upper arm and femoral artery of the upper leg or thigh. Applying pressure to an artery, also known as vascular occlusion, provides a benefit to the individual. This benefit can be achieved through exercise during vascular occlusion, but can also be achieved without concurrent exercise. In the latter case, vascular occlusion applied to patients who are unable to exercise for any variety of reasons has been shown to reduce the effects of muscular disuse atrophy and other associated muscular atrophy. Further discussion of specific studies supporting these results can be found below in the section entitled “SUPPORTING STUDIES.”

Further, low-intensity resistance exercise combined with moderate vascular occlusion provides the benefits of high-intensity training without the need to use those higher-intensity levels of resistance. Vascular occlusion is achieved by applying moderate pressure to the limbs of the individual. The moderate pressure is applied by means of a device around the limb which slightly restricts blood flow through the main arteries supplying blood to that limb. The individual performs low-intensity exercises with the device in place. The device may be removed immediately following exercise, though leaving the device in place may provide for additional benefit in situations where the individual is not wearing the device during actual exercise, such as during bed-rest. The studies discussed in the section entitled “SUPPORTING STUDIES,” have shown no identifiable negative side effects when the recommended pressure is applied over an extended period of time.

The term “muscular benefit”, as used herein, is meant to include both the increasing and maintaining of muscular size, strength, and endurance. Maintaining muscular size, strength, and endurance is needed to avoid the effects of muscular disuse atrophy. Examples of individuals affected by muscular disuse atrophy include, without limitation, patients on bed-rest, astronauts in zero- or micro-gravity, and patients recovering from surgery such as reconstruction of the anterior cruciate ligament (ACL).

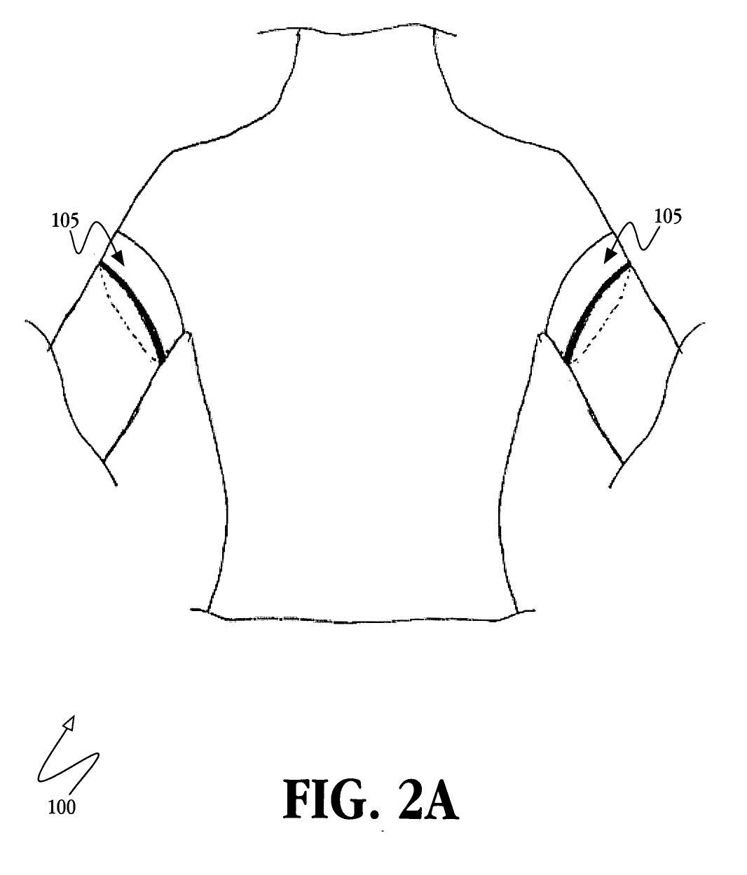

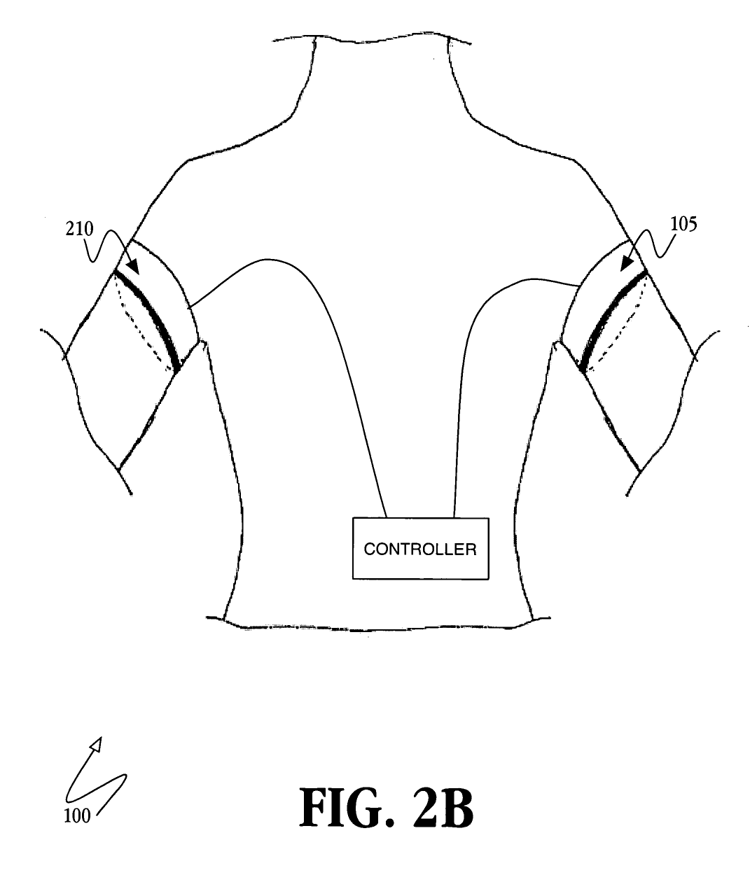

In an embodiment, the muscular benefit device 105 is configured to be applied to the upper leg or thigh of the individual. In another embodiment, the inflatable band member is configured to be applied to the upper arm of the individual. Though depicted as a single device applied to a single limb of an individual, it is understood that multiple devices may be applied to multiple limbs, increasing the efficiency of the muscular workout of the individual. Discussion of multiple devices follows with respect to FIG. 2A and FIG. 2B.

FIG. 2A is a perspective view of a system for muscular benefit using vascular occlusion, according to embodiments of the present invention. The system 200 depicted in FIG. 2A is similar to the system 100 depicted in FIG. 1 with the addition of a second muscular benefit device 210. The second muscular benefit device 205 is similar in composition to the muscular benefit device 105 depicted in FIG. 1 and described above.

In an embodiment, two muscular benefit devices 105 and 205 can be used to achieve muscular benefit concurrently on more than one limb. This is advantageous in situations where maintenance or improvement in muscular size, strength, and endurance in more than one limb is desired. Some examples where benefit to multiple limbs may be desired include, without limitation, athletes training for greater performance levels, astronauts in zero- or micro-gravity seeking to reduce the onset of muscular disuse atrophy, and individuals on extended bed-rest other than those recovering from surgery to a particular limb.

FIG. 2B is a perspective view of a system for muscular benefit using vascular occlusion, according to embodiments of the present invention. The system 210 depicted in FIG. 2B is similar to the system 200 depicted in FIG. 2A with the addition of a controller 220. In an embodiment, the controller is coupled to both muscular benefit devices 105 and 205. Use of a controller 220 to control both muscular benefit devices 105 and 205 provides an enhanced user experience to the individual as the only interaction needed to achieve the benefit of vascular occlusion is that with one device. By that one interaction, vascular occlusion occurs on both limbs concurrently.

Though depicted in FIG. 1, FIG. 2A, and FIG. 2B as being applied to an arm of the individual, the devices and systems described above are equally applicable to the leg of the individual.

As discussed above with respect to the studies, vascular occlusion provides many benefits to diverse individuals. FIGS. 1, 2A, and 2B describe, in general, muscular benefit devices 105 and 205 as applied to the limb of the individual. The next section will describe the muscular benefit device 105 in more detail and in relation to a system for muscular benefit.

Devices and Systems

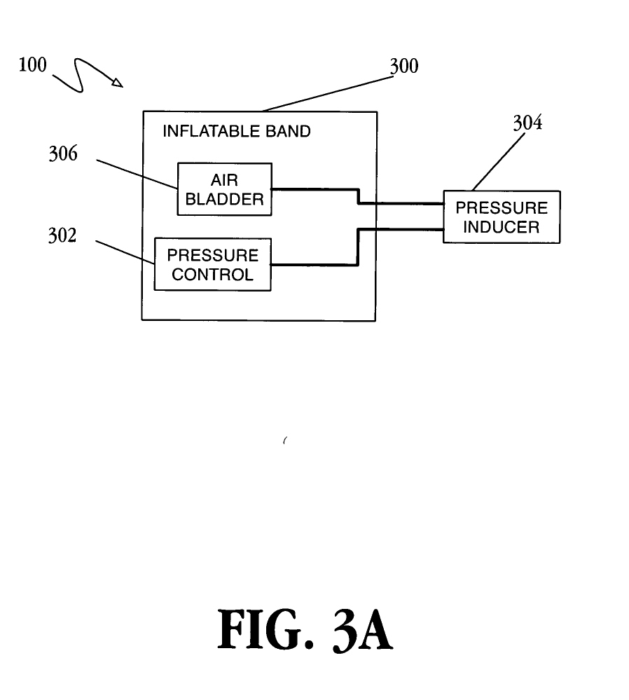

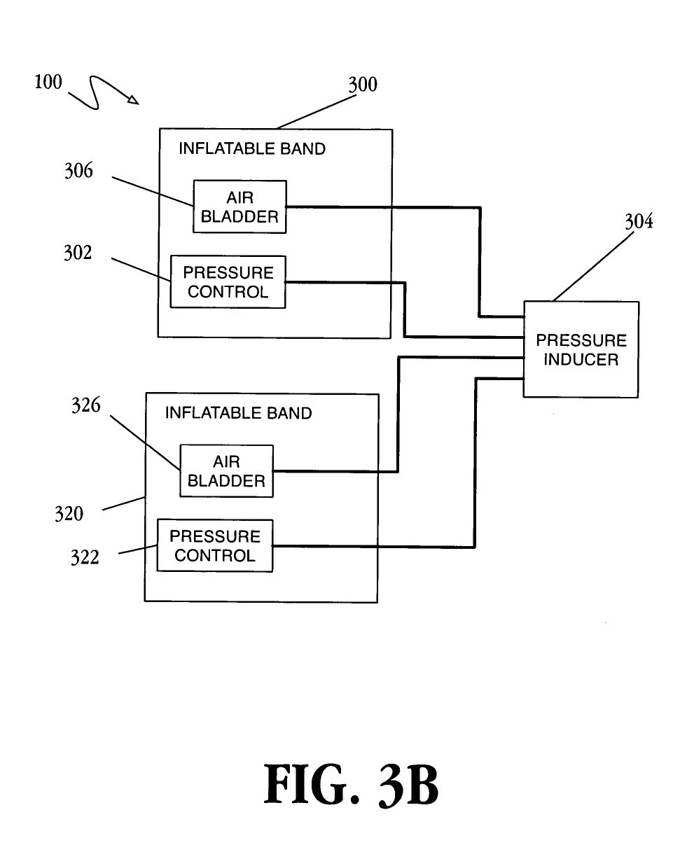

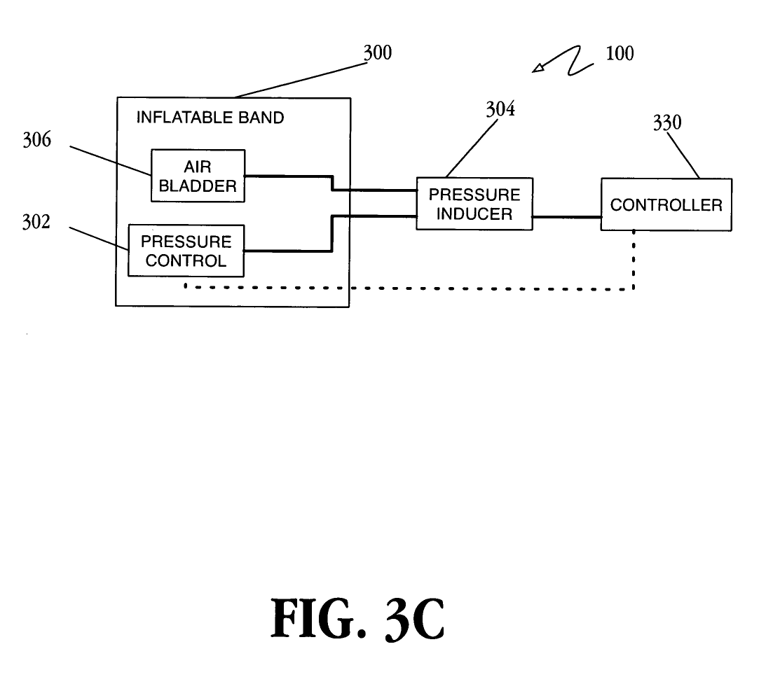

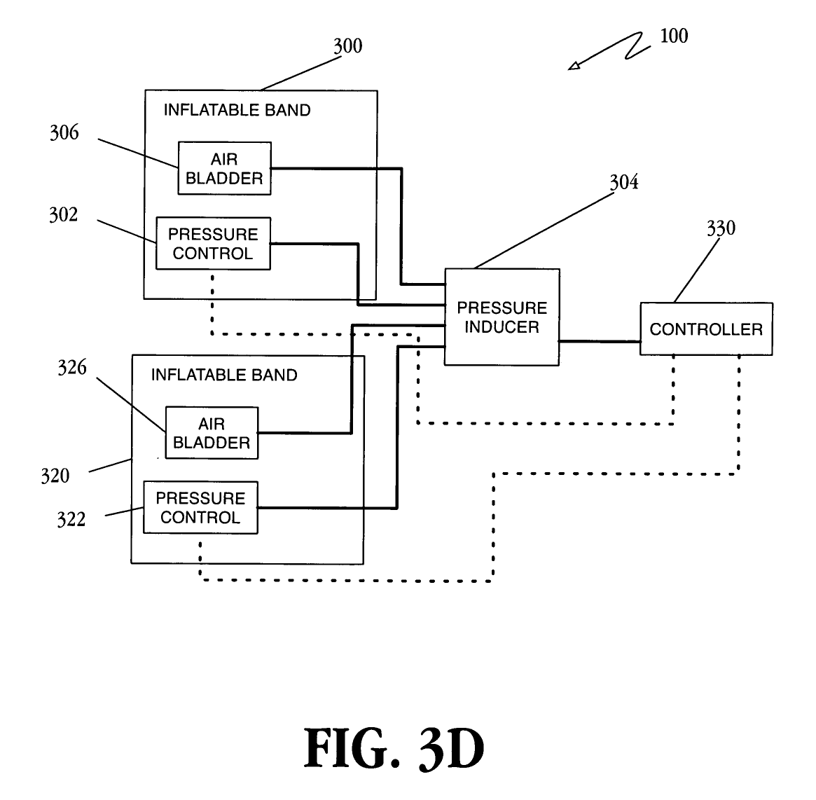

FIG. 3A is a high-level block diagram of a system, such as the system depicted in FIG. 1, for muscular benefit using vascular occlusion. In an embodiment, the muscular benefit system 100 comprises an inflatable band member 300, a pressure control device 302, and a pressure-inducing device 304. In another embodiment, the muscular benefit device further includes an air bladder 306 contained within the inflatable band member 300.

In an embodiment, the inflatable band member 300 is configured to completely encircle the limb of the individual. In one embodiment, the inflatable band member 300 is universal in size such that the same band could be used without regard to the actual size of the limb. In another embodiment, the inflatable band member 300 is adjustable in size or circumference by the individual. Means suitable for adjusting the size or circumference of the inflatable band member are well known in the art and are considered within the scope of the present discussion. In one example, the inflatable band member 300 has a hook and loop fastening system, such as Velcro®, such that the inflatable band member 300 may be adjusted by the individual to fit their limb. In another example, the inflatable band member 300 is configured similarly to a belt with any suitable fastening means. In another embodiment, the inflatable band member 300 comes in different sizes and the individual chooses which inflatable band member 300 fits best on the limb.

In another embodiment, the inflatable band member 300 further comprises an air bladder 306 within the inflatable band member 300. The air bladder 306 is configured to be inflated, causing a pressure to be applied to the limb on which the inflatable band member 300 is applied. In one embodiment, the air bladder 306 is positioned in the inflatable band member 300 such that, when inflated, pressure is applied to an artery in the limb.

In an embodiment, the pressure-inducing device 304 is coupled to the inflatable band member 300 and provides air to the inflatable band member 300 causing the inflatable band member 300 to inflate. In such an example, the inflation of the inflatable band member 300 causes a pressure to be applied to an artery on the limb of the individual. In another embodiment, the pressure-inducing device 304 is coupled to the air bladder 306 contained within the inflatable band member 300. In such an example, the pressure-inducing device 300 provides air to the air bladder 306, causing the air bladder 306 to inflate and pressure to be applied to the artery.

In an embodiment, the pressure control device 302 is coupled to the inflatable band member 300 and maintains a pressure in the inflatable band member 300. In another embodiment, the pressure control device 302 is coupled to the air bladder 306 contained in the inflatable band member 300. The pressure control device 302 includes, without limitation, a pressure relief valve configured to release air when the pressure exceeds some value.

In one embodiment, the pressure-inducing device 107 is capable of being operated manually by the individual. An example of such a device, without limitation, would be a small pressure bulb that the user would press repeatedly to inflate the inflatable band member 105 or an air bladder in the inflatable band member 105. In another embodiment, the pressure-inducing device 107 is an electronically controlled device. In such an example, the individual can set the pressure and the pressure-inducing device can inflate the inflatable band member to such a pressure. In another embodiment, the pressure-inducing device 107 and the pressure control device are combined in a single device such that the individual can activate the device and the device will inflate the inflatable band member 105 and, when the desired pressure is reached, will maintain that pressure over an extended period of time. As discussed below, maintaining the pressure on the artery over an extended period of time provides muscular benefit. As discussed below in the supporting studies, a light pressure on the artery can be maintained for several hours, or longer, with no adverse side effects.

In an embodiment, the muscular benefit device 105 is configured to be manually operated by the individual. In such an example, a device would be disposed on the inflatable band member 300 which the user operates to provide air to the inflatable band member 300. One example of such a device is the inflation mechanism discussed in U.S. Pat. No. 6,785,985 “Shoe having an inflatable bladder,” assigned to Reebok, Intl, incorporated herein by reference. Discussion of the inflation mechanism is merely illustrative and is not meant to be limiting in any manner. Any device capable of being manually operated by the individual and supplying air to the inflatable band member 300 or air bladder 306 through the manual actions of the individual is considered to be within the scope of the present discussion. One other example is a small pressure bulb disposed on the inflatable band member 300 which the individual presses repeatedly to provide air to and thereby inflate the inflatable band member 300 or the air bladder 306 in the inflatable band member 300. In another embodiment, the pressure-inducing device 107 is an electronically controlled device. Air received by the inflatable band member 300 or air bladder 306 causes a pressure to be applied to the limb on which the muscular benefit device is applied to, or more particularly to an artery within the limb. In embodiments where the individual manually provides air to the inflatable band member 300 or air bladder 306, the pressure control device 302 provides a means for the inflatable band member 300 to reach and maintain a desired pressure. In such an example, when the individual is manually providing air, the desired pressure will be reached and the pressure control device 302 will release any additional air provided to the inflatable band member 300. Through this mechanism, the inflatable band member will supply the desired pressure to the limb without regard to additional manual supply by the individual. Such operations are advantageous in that an individual not trained in the proper amount of pressure to be applied to the limb can achieve muscular benefit without the need to continually monitor what pressure is being applied.

FIG. 3B is a high-level block diagram of a system, such as the system depicted in FIG. 2A and FIG. 2B, for muscular benefit using vascular occlusion. The system 100 depicted in FIG. 3B is similar to that of FIG. 3A, with the addition of a second inflatable band member 320. The second inflatable band member is similar in construction to the inflatable band member 300 described above with respect to FIG. 3A, and includes a pressure control device 322. In another embodiment, the second inflatable band member also includes an air bladder 326. In an embodiment, the system includes two or more inflatable band members coupled to a single pressure-inducing device 304. As discussed above, the single pressure-inducing device 304 in one embodiment is configured to be manually operated by the individual. A single pressure-inducing device 304 provides the user the ability to provide air concurrently to more than one inflatable band member.

FIG. 3C is a high-level block diagram of a system, such as the system depicted in FIG. 2A and FIG. 2B, for muscular benefit using vascular occlusion. The system depicted in FIG. 3C is similar to that of FIG. 3A, with the addition of a controller device 330 and an electronically controlled pressure-inducing device 304. In an embodiment, the controller device 330 is configured to send and receive signals from the pressure-inducing device. In such an example, the pressure-inducing device 304 is configured to provide air to the inflatable band member 300 or air bladder 306 without manual intervention by the individual. The controller device 330 may include, without limitation: a selector switch whereby the individual can select the type of limb that the inflatable band member 300 is applied to; two buttons, one for activation and a second for deactivation; or any other suitable means for the user to interact with the controller. As discussed above, different limited pressures are required to be applied to the arm or leg for muscular benefit.

In one embodiment, the pressure applied to the limb of the individual when the limb is the arm, or more particularly the upper arm, is preferably in the range of about 80 to about 140 mm Hg. Most preferably, the range of pressure applied to the limb in this example is in the range of about 100 to about 115 mm Hg.

In another embodiment, the pressure applied to the limb of the individual when the limb is the leg, or more particularly the upper leg or thigh, is preferably in the range of about 180 to about 250 mm Hg. Most preferably, the range of pressure applied to the limb in this example is in the range of about 205 to about 225 mm Hg.

In one embodiment, the individual sets a pressure on the controller through any suitable means after having been directed through other means on what pressure to apply to the limb the device is worn. Other means include, without limitation, literature provided to the individual with the device. In another embodiment, the individual can select whether pressure is applied to the arms and/or legs and, more specifically, whether pressure is applied to one or both arms or, legs. After such selection, the controller will determine the proper amount of pressure to be applied.

FIG. 3D is a high-level block diagram of a system, such as the system depicted in FIG. 2A and FIG. 2B, for muscular benefit using vascular occlusion. The system in FIG. 3D is similar to that of FIG. 3C with the addition of a second inflatable band member 300. In an embodiment, a single controller device 330 is coupled to more than one inflatable band member 300 through a pressure-inducing device. In an alternate embodiment, a single controller 330 and a single pressure-inducing device 304 are coupled to more than one inflatable band member. In such an example, the functions of the pressure-inducing device 304 and the controller 330 may be combined in a single device.

In further embodiments, the devices depicted in FIG. 3A-3D may be contained within a wearable garment. Examples of wearable garments include, without limitation, pants, shorts, shirts, bodysuits, swimsuits, and the like. In such an example, the inflatable band member 300 is an integral part of the garment such that, when the garment is worn by the individual, the inflatable band member 300 is in proximity to the artery to which pressure is to be applied. In one embodiment, the inflatable band member is an integral part of the wearable garment and is not removable by the individual. In an alternate embodiment, the inflatable band member is an integral part of the wearable garment and is removable by the individual. In such an example, the individual may desire to launder the wearable garment separately. In another embodiment, the inflatable band member 300 is removably attached to the wearable garment. The term “removably attached”, as used herein, is meant to denote any suitable system or method of attaching the device to a wearable garment that provides the individual the ability to remove the device. This may include, without limitation, a hook and loop fastening system to the inside of the wearable garment such that, when worn, the inflatable band member is not visible to another individual. Additionally, the inflatable band member may be removably attached externally to the wearable garment in the area to which pressure is to be applied. Attachment to the wearable garment may be through any means suitable, such as a hook and loop fastening system. Additionally, the inflatable band member 300 may be elastic such that, when placed externally over the wearable garment, the inflatable band member 300 maintains its relative position without being removably attached to the wearable garment as with a hook and loop fastening system.

Methods

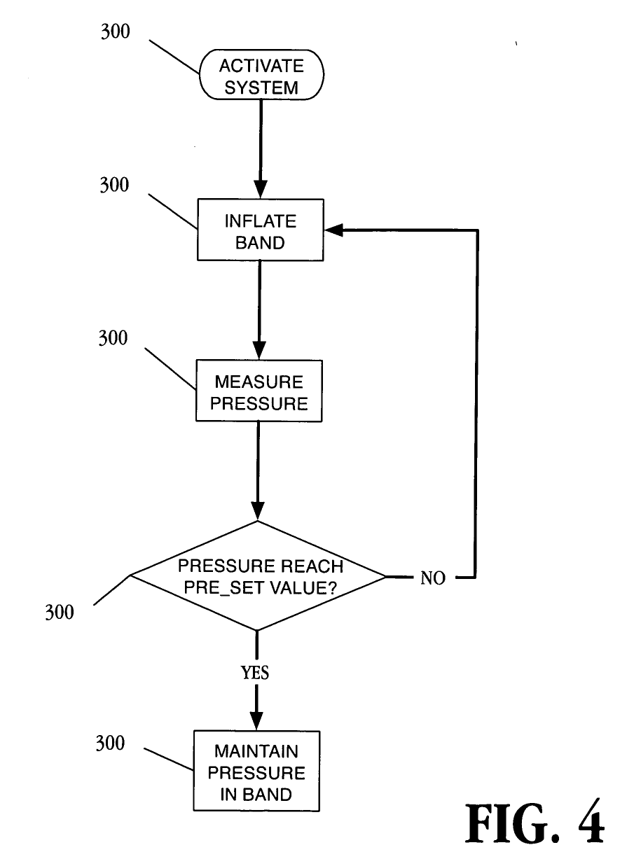

FIG. 4 is a flowchart of a method to be carried out in accordance with the embodiments of the present invention. In one embodiment, the method described herein is carried out on the systems and devices described above to facilitate muscular benefit using vascular occlusion.

At block 400, the system is activated by the individual. Following activation, at block 405, a wearable inflatable band, such as the inflatable band member 300 described above, is inflated. In an alternate embodiment, the air bladder 306 of the inflatable band member is inflated. At block 408, the pressure in the inflatable band member in the first embodiment and the air bladder in the second embodiment are measured by any means suitable. At block 410, the pressure in the wearable inflatable band is compared to some pre-set value and it is determined if the pressure has reached that value. If the pressure has reached the pre-set value, the pressure is maintained at block 415 at that value.

In an embodiment, the pressure will adjust according to the conditions present in the limb. It will be understood that the conditions of an individual's limb are not static and that air pressure in the inflatable band member will adjust to maintain the same affect on the limb. As the individual exercises, the cross-sectional area of the limb will increase (muscle getting larger). If the pressure applied to the inflatable band member was to remain static, the pressure applied to the limb would actually increase past the nominal range for muscular benefit. This may lead to an increased risk for potential side effects. In this embodiment, at block 415, the pressure may actually be reduced through any suitable means such that the actual pressure applied to the limb of the individual is maintained.

Pressure can be maintained in any suitable manner, though use of a pressure control device as described above is preferable. In the case of manual operation by an individual, the pressure control device is a pressure relief valve such that the individual can not over-pressurize the wearable inflatable band. In such an example, the operations of the pressure control device and the pressure-inducing device are separate. In the case of a system such as that depicted in FIG. 3C or FIG. 3D, the controller is coupled to both the pressure-inducing device and the pressure control device. In such a system, the controller controls the operations of both devices such that the pressure-inducing device is activated to bring the pressure to the pre-set value and then deactivated. In this example, the pressure control device may be a simple pressure sensor sending a signal to the controller through which the controller can perform the operations at block 410 and 415 by controlling the pressure-inducing device.

Though discussion is herein made to methods using a device which, when inflated, causes a pressure to be applied to the limb of an individual, and more particularly to an artery in the limb, an alternate embodiment applies pressure to the limb by means of a non-inflatable cord. In one embodiment, the non-inflatable cord is integrally contained in a wearable garment. In another embodiment, the non-inflatable cord is not contained in a wearable garment and is adapted to be applied by the individual in any suitable manner. In such an arrangement, a non-inflatable cord completely encircles the limb and, when tightened, applies pressure to the artery. In this embodiment, the individual applies the non-inflatable cord, tightens the non-inflatable cord to apply pressure to the limb, secures the non-inflatable cord to maintain pressure on the limb, and leaves the non-inflatable cord in place for an extended period of time that may be concurrent with exercise or not concurrent with exercise. Through such operations, the individual may derive the muscular benefits outlined above.

### EXAMPLE IMPLEMENTATIONS

The systems, devices, and methods discussed above facilitate muscular benefit using vascular occlusion. In this section, application of the systems, devices, and methods during specific activities will be discussed.

In an example implementation, an individual interested in improving size and strength through high-intensity resistance training would use the muscular benefit device on the limb where improvements in size and strength are sought. Through vascular occlusion of the upper arm, for example, the weight trainer can improve size and strength with less weight and fewer repetitions. As the muscle breaks down during exercise, which the systems described herein provide for, it will rebuild and, in the case of resistance exercises, will become larger and stronger.

In an example implementation, an individual interested in improving endurance through aerobic exercise would use the muscular benefit device on the limb or limbs where improvements in endurance are sought, such as the legs if the individual were a long-distance runner. The device enhances the runner's ability to sustain longer periods of muscular effort when not using the device by decreasing the time needed to reach fatigue when wearing the device. Improving endurance without the use of the device described herein would require a runner to constantly increase the distances they run.

Using the devices described herein allows shorter, more efficient exercise sessions to build up the athlete's endurance without the negative impact running longer distances can have on other areas of the body.

Though discussion is made above to athletes, the systems, devices and methods have equal application to non-athletes. For example, during the recovery from surgery to a limb, there is a period in which the individual is not able to exercise or use the muscles in the limb. Without use of the limb, the muscles in that limb will atrophy causing the individual to go through longer rehabilitation to achieve the muscular performance they had prior to the surgery. In contrast, using the devices described herein, the individual would be able to at least maintain their muscular performance during the time in which they are unable to exercise or use the limb, reducing the time required to return to the level of muscular performance they had before the operation. Alternatively, during long periods of bed-rest, muscular atrophy takes place. Using the devices described herein, the individual could maintain the pressure levels described herein on their limbs as a means of decreasing the effects of the atrophy caused by bed-rest.

Another example implementation would be with astronauts in micro- or zero-gravity environments. Much research has been conducted measuring the long-term affects of these environments and the research shows that exercise will reduce the effects of muscular disuse atrophy. Usage of the devices described herein will increase the benefits of such exercise, allowing the astronaut to realize as little a reduction in muscular performance as possible. The case of astronauts in micro- or zero-gravity is also similar to those of a patient on bed-rest who is unable to use or exercise their limbs and the discussion above is equally applicable here.

Another example implementation would be individuals who wish to get the benefit of exercise without the need for actual exercise. Using the devices and methods outlined here, the individual applies the device and a limited pressure to their limb, either arm or leg singly or in combination with other vascular occlusion devices. The limited pressure slightly restricts blood flow and thereby causes muscular benefit. The individual could be bed-ridden and, through the use of the devices and methods outlined here, would reduce the effects of that bed-ridden state, e.g. muscular disuse atrophy.

Yet another example implementation involves individuals afflicted with peripheral arterial disease (PAD), also known as peripheral vascular disease. PAD refers to diseases of blood vessels outside the heart and brain. It's often a narrowing of vessels that carry blood to the legs, arms, stomach, or kidneys. PAD includes both functional PAD, which does not have an organic cause, and organic PAD, which is caused by structural changes within the blood vessels. One example of functional PAD is Raynaud's disease, which can be triggered by cold temperatures, emotional stress, or smoking. In its early stages, PAD leads to increased fatigue during normal activity. In patients with PAD, high-intensity resistance or endurance exercise may have very detrimental side effects such as an increased risk of stroke and heart attack. Using the devices and methods outlined herein, the patient can go about their normal activity and, with limited pressure applied to the limb, can slow the onset of fatigue when the device is not worn by improving muscular size, strength, and endurance when it is worn, without the need for intensive and potentially life-threatening exercise. In such an example, the patient could wear any one of the devices outlined above and, combined with either low-intensity exercise or in the absence of exercise, could achieve muscular benefit such that when the patient is not wearing the device they would experience less fatigue and could perform normal activities for longer periods of time, thereby increasing their quality of life.

Supporting Studies

Several studies support the claim that vascular occlusion during exercise or in the absence of exercise results in muscular benefit to the individual. Discussion of those studies is provided here.
Takarada Y, Nakamura Y, Aruga S, Onda T, Miyazaki S, Ishii N., “Rapid increase in plasma growth hormone after low-intensity resistance exercise with vascular occlusion,” J Appl Physiol. January 2000;88(1):61-5, incorporated herein by reference.

Hormonal and inflammatory responses to low-intensity resistance exercise with vascular occlusion were studied. The exercise session started 5 min after the resting blood sample was drawn. After the exercise sessions, the occlusion was released and blood samples were obtained at 0 (immediately after exercise), 15, 45, and 90 min, and at 24 h. to record plasma concentrations of lactate (La), growth hormone (GH), and norepinephrine (NE).

The study showed that resistance exercise combined with vascular occlusion, even at an extremely low-intensity, causes enhanced muscular electrical activity and endocrine responses. The increase in plasma GH concentration was much greater in magnitude than that reported to occur after the typical exercise (high-intensity, short rest period) widely used for gaining muscular size. Such an effect would not be associated with serious tissue damage, because both plasma markers for muscular damage, creatine phosphokinase (CPK) activity and oxidative stress (LP concentration), did not increase considerably. However, slight elevation in the plasma concentration of interleukin-6 (IL-6) suggests finer micro-damage occurring within vascular walls and/or muscle tissue. The study also revealed that the peak concentration of La after the exercise with occlusion was twice as large as that after the exercise without occlusion.

It was concluded that high-intensity exercise to achieve recruitment of fast-glycolytic fibers is not required for regional accumulation of metabolites when muscles are forced to contract in a hypoxic condition and the metabolite clearance is simultaneously suppressed. Such a condition is satisfied when low-intensity exercise is combined with vascular occlusion. The relative electrical activity of the muscle during the exercise with occlusion was ˜1.8 times as large as that during the exercise without occlusion (P<0.01), even though both the force generated and the mechanical work produced were to be the same between these two kinds of exercise.

Even more substantial, the concentration of GH reached a level of ˜290 times as high as that of the resting level 15 min after the exercise. The results suggest that extremely light resistance exercise combined with occlusion greatly stimulates the secretion of GH through regional accumulation of metabolites without considerable tissue damage. This magnitude of increase in GH concentration was larger by a factor of ˜1.7 than that reported by Kraemer et al. for high-intensity resistance exercise with a short rest period (typical bodybuilding routine), indicating that the exercise with occlusion can provoke strong endocrine responses even at an intensity of just 20% of someone's one repetition maximum. Plasma concentrations of testosterone and insulin-like growth factor I (IGF-I) have been shown to behave in a substantially similar manner as well.
Takarada Y, Takazawa H, Sato Y, Takebayashi S, Tanaka Y, Ishii N., “Effects of resistance exercise combined with moderate vascular occlusion on muscular function in humans,” J Appl Physiol. June 2000;88(6):2097-106, incorporated herein by reference.

In this study, a low-intensity resistance exercise training regimen with moderate vascular occlusion caused a marked muscular hypertrophy. Before and after exercise, arterial blood flow and plasma lactate concentration were measured. The exercises used were low-intensity with occlusion (LIO) and low-intensity without occlusion (LI) for the occlusive training and normal training groups, respectively.

In the low-intensity exercise, post-exercise hyperemia increased with occlusion pressure and tended to be larger than that in the high-intensity exercise at an occlusion pressure of 100 mmHg. Plasma lactate concentrations were measured immediately after the exercises at high- and low-intensities, either with or without occlusion at 100 mmHg. No considerable difference was observed between plasma lactate concentration measured after the high-intensity exercise and that after the low-intensity exercise when both exercises were performed without occlusion. When exercise was combined with occlusion at 100 mmHg, however, plasma lactate concentration after the low-intensity exercise dramatically increased from that without occlusion, exhibiting a sharp contrast to that after the high-intensity exercise, which was almost unchanged with the increase in occlusion pressure.

Among aged populations, the weakening of muscles in the lower extremities, in particular, gives rise to serious problems with mobility and quality of life. Specifically, the inability of many to stand up and the increased risk of lethal injuries associated with falls. Post-menopausal women have the additional risk of osteoporosis. One of the most effective ways to counter these problems is resistance exercise. However, resistance exercise, particularly in older people, carries with it its own risks. This study showed that low-intensity exercise combined with vascular occlusion is useful for accelerating the recovery of muscle strength in patients and in aged people. Additionally, as was shown in other studies, vascular occlusion with no exercise for patients unable to perform exercise, or for older people in which exercise presents other risks, does have a positive effect on muscular performance.
Takarada Y, Takazawa H, Ishii N., “Applications of vascular occlusion diminish disuse atrophy of knee extensor muscles,” Med Sci Sports Exerc. December 2000;32(12):2035-9, incorporated herein by reference.

Muscles adapt themselves rapidly to the environment they are in. In environments of strong mechanical stress, such as resistance exercise, they respond with muscular hypertrophy. In environments such as space flight or immobilization following surgery, they respond with muscular disuse atrophy. With respect to recovery from knee surgery, reducing muscular disuse atrophy is important, as any reduction will result in a shorter rehabilitation period for the patient. In other individuals, such as the elderly, age-related atrophy of the knee extensor muscles results in the inability to stand up. Low-intensity exercise combined with vascular occlusion has been previously shown to have positive results. The present study examined whether vascular occlusion without any concomitant exercise would have any effect in diminishing post-operative muscular disuse atrophy.

Usage of vascular occlusion without exercise was shown to have a definite positive effect on the retention of muscular size and strength during the initial periods after surgery. In combination with low-intensity exercise during the subsequent period of rehabilitation, a marked shortening of the period required for a full recovery was observed.

Vascular occlusion effectively diminishes the disuse atrophy of muscles during a period following surgery in which exercise is not possible. It is also useful for improving muscular function in patients that are bed-ridden for whatever reason.
Takarada Y, Sato Y, Ishii N., “Effects of resistance exercise combined with vascular occlusion on muscle function in athletes,” Eur J Appl Physiol. February 2002;86(4):308-14, incorporated herein by reference.

The effects of resistance exercise combined with vascular occlusion on muscle function were investigated in highly trained athletes. Elite rugby players (n=17) took part in an 8 week study involving the training of the knee extensor muscles using low-intensity exercise equal to about 50% of the subject's one repetition maximum combined with an occlusion pressure of about 200 mmHg (LIO, n=6), low-intensity exercise without the occlusion (LI, n=6), and no exercise training (untrained control, n=5). The exercise in the LI group was of the same intensity and amount as in the LIO group. The LIO group showed a significantly larger increase in isokinetic knee extension torque than that in the other two groups (P<0.05) at all the velocities studied. On the other hand, no significant difference was seen between LI and the control group. In the LIO group, the cross-sectional area of knee extensors increased significantly (P<0.01), suggesting that the increase in knee extension strength was mainly caused by muscle hypertrophy. The dynamic endurance of knee extensors estimated from the decreases in mechanical work production and peak force after 50 repeated concentric contractions was also improved after LIO, whereas no significant change was observed in the LI and control groups. The results indicated that low-intensity resistance exercise causes, in almost fully trained athletes, increases in muscle size, strength and endurance, when combined with vascular occlusion.

The present study shows that low-intensity resistance exercise combined with vascular occlusion causes muscular hypertrophy, increased strength, and endurance. The study was conducted on elite rugby players but the fact that these athletes were fully trained yet still experienced marked increases in muscle size, strength, and endurance suggests using low-intensity resistance exercise combined with vascular occlusion would yield even greater benefit to non-fully trained individuals.
Ohta H, Kurosawa H, Ikeda H, Iwase Y, Satou N, Nakamura S., “Low-load resistance muscular training with moderate restriction of blood flow after anterior cruciate ligament reconstruction,”Acta Orthop Scand. February 2003;74(1):62-8, incorporated herein by reference.

New techniques of anterior cruciate ligament (ACL) reconstructive surgery have permitted patients to begin their rehabilitation earlier, allowing them a faster return to athletics and daily living. However, the issue is how quickly the patient can return to athletic activities. In all of the methods of ACL reconstruction, such training can not begin until the ligament has healed sufficiently to bear the stress load of the training. Therefore, the patient will experience atrophy of the muscles around the knee, in particular the knee extensor muscle, which causes reduced muscular strength. Prevention or reduction of this atrophy is desirable as it will allow for an earlier recovery of muscular strength and an earlier return to athletic activities.

The present study examined the proposition that low-load resistance training with vascular occlusion during the early rehabilitation period before the ligament can bear the stress load of athletic activities and training, improves muscular strength and size.

In the study, subjects who used vascular occlusion during low-load resistance training showed significant recovery of muscular strength compared to those subjects who did not. The knee flexor muscle torque also showed significant recovery of muscular strength.

Vascular occlusion during low-load training during the early rehabilitation from ACL reconstruction is effective in reducing the effects of atrophy and shortening the amount of time it takes the patient to return to their normal level of activity. Additionally, the study suggests the benefits patients received following ACL reconstruction are equally applicable to the rehabilitation of any atrophied muscles. In general, use of vascular occlusion during recovery from any surgery that would restrict the movement and activity of any limb would be very useful to the patient.
Moore D R, Burgomaster K A, Schofield L M, Gibala M J, Sale D G, Phillips S M., “Neuromuscular adaptations in human muscle following low-intensity resistance training with vascular occlusion,” Eur J Appl Physiol. August 2004;92(4-5):399-406, incorporated herein by reference.

Resistance exercise can result in both potentiating and fatiguing responses. The purpose of this investigation was to study the acute neuromuscular responses following low-intensity resistance training with vascular occlusion. Some previous studies have shown that resistance exercise training with venous occlusion causes an enhanced hypertrophy in human muscles. The OCC (with vascular occlusion) stress might be expected to induce metabolic alterations that are consistent with compromised oxygen delivery rather than an increase in strength per se.

In the present investigations, the authors have studied the effect of low-intensity training (8 weeks) with vascular occlusion (OCC) and also without vascular occlusion (CON) on neuromuscular changes in the elbow flexors of eight previously untrained men. The studies revealed that there is an increase in strength and whole muscle cross-sectional area after low-intensity resistance training with vascular occlusion (OCC). The post-activation potentiation significantly increased by 51% in OCC (P<0.05) and was not changed in CON.

The results concluded that the low-intensity resistance training in combination with vascular occlusion produces an adequate stimulus for increasing muscle strength without the need for large resistive loads. Additionally, it causes changes in the indices of neuromuscular functions such as depressed resting twitch torque and enhanced post-activation potentiation.

It is to be understood that the above description is intended to be illustrative, and not restrictive. Many other embodiments will be apparent to those with skill in the art upon reading and understanding the above description. It should be noted that embodiments discussed in different portions of the description or referred to in different drawings may be combined to form additional embodiments of the present application. The scope of the invention should, therefore, be determined with reference to the appended claims along with the full scope of equivalents to which such claims are entitled.

What is claimed is:

1. A vascular occlusion device to provide limited pressure to a limb of an individual, the device comprising:
an inflatable band member to completely encircle a limb of an individual, the inflatable member to be inflated and cause to be effected a pressure on the limb;
a pressure-inducing device coupled to the inflatable band member to provide air to the inflatable band member causing the inflatable band member to inflate and effect the pressure on the limb; and
a pressure control device coupled to the inflatable band member to maintain the pressure on the limb.

2. The vascular occlusion device of claim 1, wherein the inflatable band member includes an air bladder to be inflated.

3. The vascular occlusion device of claim 2, wherein the air bladder is disposed along the entirety of a length of the inflatable band member.

4. The vascular occlusion device of claim 2, wherein the air bladder is contained within the inflatable band member, but is not disposed along the entirety of a dimension of the inflatable band member.

5. The vascular occlusion device of claim 1, wherein the pressure-inducing device is a manual pump device attached to the inflatable band member, the manual pump device to be controlled by the individual.

6. The vascular occlusion device of claim 1, wherein the pressure-inducing device is an electronically controlled pump device.

7. The vascular occlusion device of claim 1, further comprising:
a pressure sensor; and
a controller coupled to the electronically controlled pump device and the pressure sensor to control the operations of the electronically controlled pump device based on the readings from the pressure sensor.

8. The vascular occlusion device of claim 7, wherein the inflatable band member, pressure-inducing device, pressure sensor, and controller are integrally contained in a wearable garment.

9. A vascular occlusion system to provide limited pressure to one or more limbs of an individual as a means of facilitating muscular benefit in the one or more limbs of the individual, the system comprising:
one or more wearable inflatable band members, each of the one or more wearable inflatable band members adapted to completely encircle one of a plurality of limbs of an individual, and to be inflated and cause a pressure to be applied to the one of the plurality of limbs;
one or more pressure-inducing devices coupled to the one or more wearable inflatable band members to provide air to the one or more wearable inflatable band members causing the inflatable band to inflate and effect the pressure on the limb; and
one or more pressure control devices coupled to the one or more wearable inflatable band members.

10. The vascular occlusion system of claim 9, wherein the one or more pressure control devices is one or more pressure sensors, each of the one or more pressure sensors to read a pressure in the one or more wearable inflatable band members.

11. The vascular occlusion system of claim 10, further comprising a controller coupled to the one or more wearable inflatable band members, the one or more pressure-inducing devices, and the one or more pressure sensors, the controller to control the operations of the pressure-inducing devices based on the pressure readings received from the one or more pressure sensors.

12. The vascular occlusion system of claim 11, wherein the controller is configured to maintain a steady pressure on the limb of an individual.

13. A method of vascular occlusion of a limb to stimulate muscular benefit, the method comprising:
inflating a wearable band;
determining if a pressure in the wearable inflatable band has reached a pre-set value; and
maintaining the pressure in the wearable inflatable band at the current pressure if the pressure in the wearable inflatable band has reached the pre-set value, where the pressure is maintained over an extended period of time.

14. The method of claim 13, wherein pre-set value is between 80 and 140 mm Hg.

15. The method of claim 13, wherein pre-set value is between 180 and 250 mm Hg.

16. A method of vascular occlusion of a limb to stimulate muscular benefit, the method comprising:
applying a non-inflatable cord to a limb, wherein the non-inflatable cord is integrally contained in a wearable garment and the non-inflatable cord is adapted to be secured after tightening;
tightening the non-inflatable cord until a pressure is applied to the limb; and
securing the non-inflatable cord such that the pressure is maintained over an extended period of time.

17. The method of claim 16, wherein the wearable garment is a shirt and a non-inflatable cord is integrally contained in the upper portion of each sleeve of the shirt.

18. The method of claim 16, wherein the wearable garment is a pair of pants and a non-inflatable cord is integrally contained in the upper portion of each leg of the pair of pants.

19. The method of claim 16, wherein the wearable garment is a bodysuit and a non-inflatable cord is integrally contained in one or more of the limbs of the bodysuit.

20. The method of claim 16, wherein the wearable garment is a pair of shorts and a non-inflatable cord is integrally contained in each of the legs of the pair of shorts.
